(19) United States
(12) Patent Application Publication
(10) Pub. No.: US 2007/0073194 A1
(43) Pub. Date: Mar. 29, 2007

(54) Method for measuring rotation angle of vertebral axial

(75) Inventors: Jian-Horng Chen, Nantou City (TW); Wei-Min Chi, Longjing Shiang (TW)

Correspondence Address: LADAS & PARRY, 26 WEST 61ST STREET, NEW YORK, NY, 10023, US

(73) Assignee: CHUNG SHAN MEDICAL UNIVERSITY

(21) Appl. No.: 11/438,100

(22) Filed: May 19, 2006

(30) Foreign Application Priority Data
Sep. 15, 2005 (TW) 094131747

(51) Int. Cl. A61B 5/103

(52) U.S. Cl. 600/594

(57) ABSTRACT

The invention relates to a method for measuring vertebral axial rotation comprising: obtaining an image of a vertebra to be measured; determining centers of ellipses of pedicles of the vertebra projected on the image; measuring a distance between the centers of the ellipses; measuring a distance between a center of one of the pedicles and a medial axis of the vertebra; obtaining at least one shape parameter of the vertebra; and calculating an axial rotation angle of the vertebra according to the shape parameter and the measured distances. Machine-readable medium which can execute a method of the invention is also provided.

### FIELD OF THE INVENTION

The invention relates to a method for measuring a spinal rotation angle, and more particularly to a method for measuring the axial rotation angle of a vertebra.

### BACKGROUND OF THE INVENTION

Scoliosis is a three-dimensional deformity of the spinal column, generally meaning displacement and/or rotation of spinal segments from normal positions. Measuring the rotation angles of the spinal segments is important for observing the progress of scoliosis, operative planning and correcting these spinal columns. To determine the degree of deformity of the scoliosis, the deformation on coronal plane and sagittal plane can be measured easily and precisely through utilizing the anteroposterior view (AP-view) and lateral view X-ray film, but the rotation of a spinal segment on the transverse plane is difficult to assess. Although computed tomography (CT) technology is currently widely applied to measuring spinal deformities, and can obtain accurate measurements, the subject must have a supine position when shooting the pictures of the cross sections of the spinal segments resulting from the natural curve (e.g., lordosis and kyphsis) of the spinal column. However, the supine position reduces the effect of the gravitational force and the mechanical effect of the asymmetry of both lower limbs, such as leg length inequality. Therefore, the CT is not capable of depicting the curve of the spine and the displacement of spinal segments accurately when the subject is in a supine position. Another significant disadvantage of CT, apart from its high cost, is patient exposure to the radiation. In addition, general medical image systems obtain medical images of a patient from an image database. Only the planar data, such as length, area, and angle, can be measured by observing the images of organs in these medical images.

Other planar information, such as the cross section views, cannot be obtained in the same manner. Therefore, it is necessary to provide a medical image system and a related method for measuring the rotation angle of the spinal column with an X-ray film.

From 1948, some methods for estimating the rotation angle of the spinal column with the projections of the spinous process, the transverse process, the intervertebral foramen and the pedicle on X-ray film were published. In 1948, Cobb first proposed a method of assessing the rotation angle of a vertebra. The method proceeds based on the linear offset of the spinous process relative to the position of the vertebral body on X-ray film. The degree of rotation from normal to maximal position is expressed by ‘0’ to ‘++++’. However, the relationship between the number of ‘+’ and the actual degree of rotation is not reported. To overcome the shortage of the method proposed by Cobb, in 1969, Nash and Moe proposed that the relative position of the pedicle in relation to the vertebral body on the X-ray film could be utilized to represent the degree of rotation of a spinal segment. Since the precision of the measured result is affected by the displacement of the projection of the pedicle being non-linear relative to the rotation of the spinal segments, this method is still under consideration.

Since it causes more error to estimate the rotation of a single spinal segment, Fait and Janovec estimated a segment's rotation angle according to trigonometric relationships. They built an ideal rotation module of the spinal segments, wherein a half cyclic is utilized to imitate the front part of the vertebral body, a rectangle is utilized to imitate the rest of the vertebral body, and the edge of the rectangle denotes the pedicle. The distance between the pedicle at the convex side and the edge of the vertebral body is a, and the full width of the vertebral body is b. An approximate rotation angle is obtained after using a table with the ratio of a/b. In 1976, Benson considered that errors of calculating the rotation angle based on the position of the pedicle in an X-ray film resulted from: (1) significant changes in the shape of all vertebrae; (2) differences between the actual pedicle and pedicle images; (3) inclination of the vertebra on the sagittal plane. With an increasing vertebral rotation angle, the projected contour of the vertebral body changes, which results in some offset of the borders. Neither of these methods is completely satisfactory; however, they effectively describe the relationship between vertebral rotation and displacement of the pedicle or spinous process. In 1977, Coetsier et al. utilized the position of two pedicles and width of the vertebral body to calculate the rotation angle. However, the accuracy of this method is questioned.

In 1981, Perdriolle and Vidal created a ‘torsionmeter’ which can display vertebral rotation angles using the lateral edge of a vertebral body and the position of the middle point of the pedicle shadow on the convex side. However, this method produced errors increasing with the rotation angle.

In 1986, Stokes et al. developed a method that calculates the rotation angles of the spinal segments through utilizing the displacement of the spindle. In this method, it is necessary to take an AP-view X-ray film and an oblique X-ray film by 45 degrees, and mark six points. Russell et al. reported that the method proposed by Stokes was the least accurate of all methods and had a very complex analytical system.

In analyzing various techniques mentioned above, each technique has at least one of the following drawbacks: (1) the measured result is not a quantized angle; (2) the precision of the calculated rotation angle is not high enough; (3) with an increasing vertebral rotation angle, the error of the measured result increases; (4) it is inconvenient to proceed with the estimation procedure with two X-ray films.

Additionally, all known medical apparatuses are utilized to measure planar data, such as length, area, and angle, by observation of the AP-view X-ray film of a patient from an image database. Other information, such as the cross section view, cannot be obtained through utilizing the medical apparatus mentioned above.

The disadvantage of the techniques mentioned above is caused by: (1) the improperly selected feature point; (2) supposing that the elliptical vertebral body is a cylinder; and (3) lacking a proper analyzing technique. Therefore, prior medical apparatus lack the ability of analyzing the information of the transverse plane through utilizing the image of the coronal plane.

### SUMMARY OF THE INVENTION

The present invention provides a method for measuring vertebral axial rotation rapidly, easily and precisely.

According to an embodiment of the present invention, a method for measuring vertebral axial rotation is disclosed. The method comprises: obtaining an image of a vertebra to be measured; determining centers of ellipses of pedicles of the vertebra projected on the image; measuring a distance between the centers of the ellipses; measuring a distance between a center of one of the pedicles and a medial axis of the vertebra; obtaining at least one shape parameter of the vertebra; and calculating an axial rotation angle of the vertebra according to the shape parameter and the measured distances.

According to another embodiment of the present invention, a machine-readable medium embodied thereon a program configured to cause a machine to measure vertebral axial rotation is also provided. The program comprises code segments for causing the machine to: obtain an image of a vertebra to be measured; determine centers of ellipses of pedicles of the vertebra projected on the image; measure a distance between the centers of the ellipses; measuring a distance between a center of one of the pedicles and a medial axis of the vertebra; obtain at least one shape parameter of the vertebra; and calculate an axial rotation angle of the vertebra according to the shape parameter and the measured distances.

The other objects and achievements of the present invention will become apparent through the description of the present invention and the claims, with reference to the accompanying drawings, and the present invention will be generally understood.

### BRIEF DESCRIPTION OF THE DRAWING

FIG. 1a and FIG. 1b are schematic diagrams of a spinal segment before and after rotation.

FIG. 2 is a schematic diagram illustrating a projected relationship of FIG. 1a and FIG. 1b.

FIG. 3 is a flow chart of the method for measuring the rotation angle of the vertebral body according to an embodiment of the present invention.

FIG. 4 is a functional block diagram of an apparatus according to an embodiment of the present invention.

FIG. 5 is a schematic diagram illustrating the arrangement of an apparatus according to an embodiment of the present invention.

FIG. 6 is a schematic diagram of a cadaver spine rotation-fixation device.

FIG. 7 is a schematic diagram illustrating how to measure the actual rotation angles with CT images.

FIG. 8 is a curve illustrating the relation between the estimated rotation angle θ_{X} and the iteration times.

FIG. 9a to FIG. 9d are curves illustrating the relation between the actual rotation angle θ_{CT} and the estimated rotation angle θ_{X}.

In all of the above accompanying drawings, the same referential numerals are used to indicate the same, similar, or corresponding characteristics or functions.

### DETAILED DESCRIPTION OF THE INVENTION

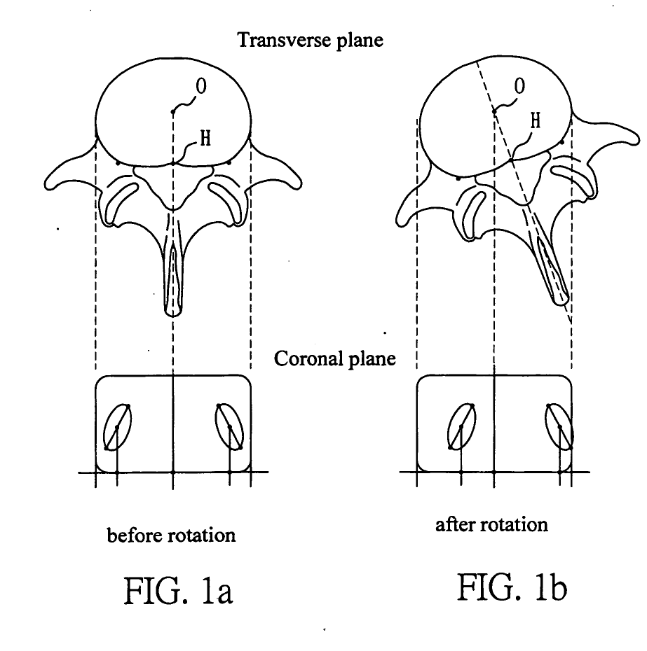

Please refer to FIG. 1a and FIG. 1b. FIG. 1a and FIG. 1b are schematic diagrams of a vertebra (or spinal segment) before and after rotation. The point H at the middle of the vertebral foramen near the vertebral body was previously considered as the rotation center. When the spinal segment rotates, it is discovered that the pedicle position is displaced relative to the vertebral body by observing an AP-view X-ray image of the spinal segment. As shown in FIG. 1a and FIG. 1b, each pedicle is roughly represented by an oval shadow. The oval's border close to the vertebral body center is considered as the inner side, and the border close to the lateral side edge of the vertebral body is considered as the outer side.

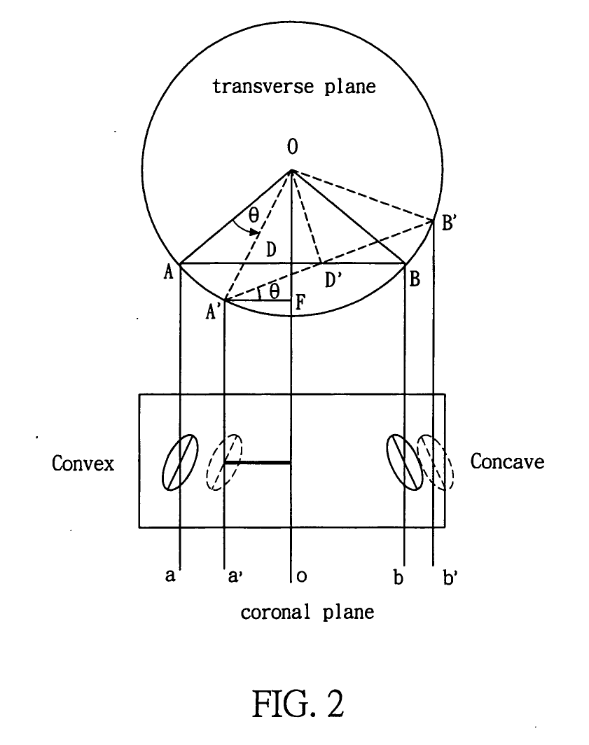

Please refer to FIG. 2. FIG. 2 is a schematic diagram illustrating a projected relationship of FIG. 1a and FIG. 1b, wherein FIG. 1a and FIG. 1b are combined herein, and the center points, O, of the vertebral bodies are superimposed. The point O is the center point of a vertebral body, and the midpoint of the connection between the cranial and caudal parts of the oval shadow denotes the position of the pedicle. As depicted in FIG. 2, letters A and B indicate the positions of the left and right pedicles before vertebral rotation, respectively, and the positions of these pedicles after rotation are marked as A′ and B′. The rotation angle θ can be represented as θ=∠AOA′. Furthermore, let the projections of two pedicles (before and after rotation) and the center of the vertebral body on the film be denoted by a, b, a′, b′ and o, respectively. Additionally, D is set at the midpoint of A̅B̅, and a straight line, A̅F̅, is drawn perpendicular to O̅o̅ with point F located at the intersection of the two lines. Based on trigonometric relationships, the following equations are obtained:

$$\begin{matrix}θ = ∠ AOD - ∠ {A}^{′} OF & Equation ( 1 ) \\ ∠ {A}^{′} OF = {sin}^{- 1} \frac{\overline{{A}^{′} F}}{\overline{{OA}^{′}}} & Equation ( 2 )\end{matrix}$$

Moreover, the distance between the vertebral body center O and the pedicle at the convex side is:

$$\overline{{OA}^{′}} = \overline{OA} = \frac{\overline{AD}}{sin ∠ AOD} \qquad Equation ( 3 )$$

Since

$$\overline{AD} = \frac{1}{2} \overline{AB} ,$$

let the actual distance between the two pedicles be A̅B̅= a̅b̅=w, then Equation (3) can be rewritten as:

$$\overline{{OA}^{′}} = \overline{OA} = \frac{\overline{AB}}{2 sin ∠ AOD} = \frac{w}{2 sin ∠ AOD} \qquad Equation ( 4 )$$

In Equation (4), ∠AOD is correlated with the vertebral body shape, which is determined by the ratio of A̅D̅ and O̅D̅. Additionally,

$$\frac{\overline{AD}}{\overline{OD}} = η$$

denotes the shape parameter of the vertebral body.

It should be noted that an AP radiograph taken in a standing position only obtains a coronal plane image (e.g., lower part of FIG. 2). Consequently, without other clues in a film, the shape parameter η for every vertebral body should be obtained from statistical data. Stokes et al. obtained statistical means of the width-to-depth values for vertebral bodies L1-L4 as shown in Table 1; half of the width-to-depth value is the shape parameter η in this study. Thus, ∠AOD=tan⁻¹ η is derived.

TABLE 1
Shape parameter of the vertebral body and corresponding ∠AOD

| | Vertebra | | | | |
|---|---|---|---|---|---|
| | L1 | L2 | L3 | L4 | L5 |
| Shape | 0.97 | 0.92 | 1.04 | 1.25 | — |
| parameter | | | | | |
| η(statistical | | | | | |
| value) | | | | | |
| ∠AOD ( ) | 44.1 | 42.6 | 46.1 | 51.3 | — |

When an AP-view X-ray film of a spinal segment is obtained, a̅′̅o̅ and a̅′̅b̅′̅ can be evaluated. As shown in FIG. 2, it is obvious that a̅′̅o̅= A̅′̅F̅. If a̅′̅b̅′̅=w′, the initial value of w is set to be w′, and thus an approximate value of O̅A̅′̅ is obtained according to the Equation (4). It should be noted that, in the present embodiment, the value of ∠AOD is obtained according to the Table 1 without referring to cross section views of Computed Tomography (CT) or Magnetic Resonance Imaging (MRI). Furthermore, an approximate value of ∠AOF′ is obtained by calculating Equation (2) with an approximate value of O̅A̅′̅ and the estimated value of A̅′̅F̅, and thus an approximate value of the rotation angle θ is obtained by calculating Equation (1).
A̅′̅B̅′̅ cos θ= a̅′̅b̅′̅ Equation (5)
A̅B̅= A̅′̅B̅′̅=w Equation (6)
According to Equations (5) and (6), the value of w can be adjusted, and then the steps mentioned above are repeated until the value of θ is smaller than a predetermined value and thus is convergent. The procedure of performing the steps mentioned above will be described as follows.

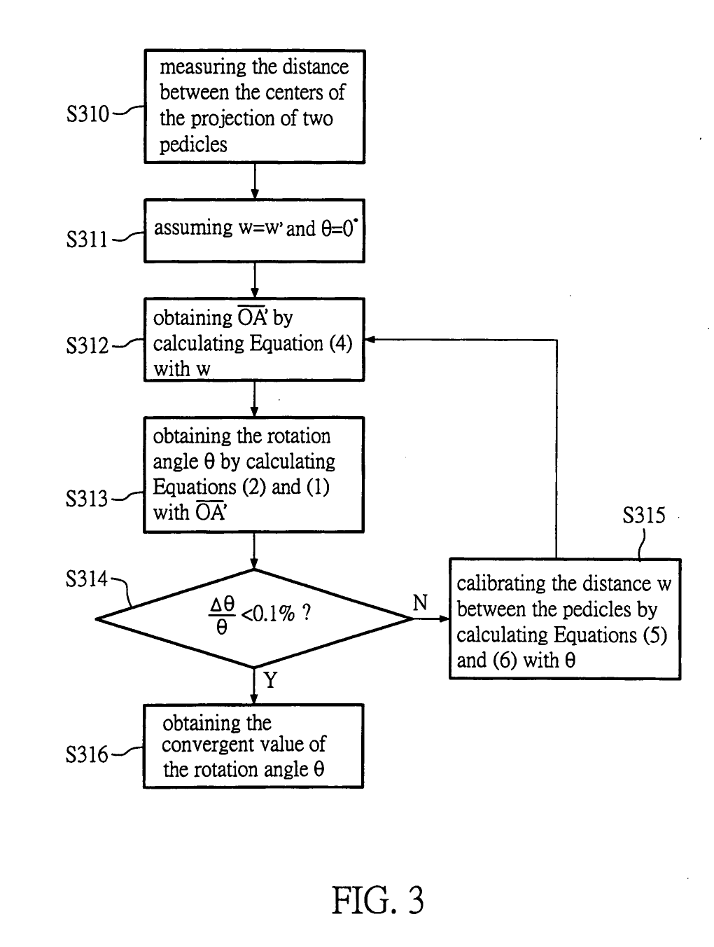

FIG. 3 is a flow chart of the method for measuring the rotation angle of the vertebral body according to an embodiment of the present invention. Firstly, the method measures the distance w between the two pedicles (step S310), assigns the initial value of θ to zero, and assigns the value of w′ equal to w (step S311). Secondly, the method calculates the value of O̅A̅′̅ by calculating Equation (4) with w (step S312). Thirdly, the method calculates the rotation angle θ′ by calculating the Equations (2) and (1) with the value of O̅A̅′̅ (step S313). Next, if the ratio of the difference between θ and θ′ (i.e., Δθ) to the value θ is smaller than a predetermined value (e.g., 0.1), the method proceeds to step S315. In step S315, the value of the rotation angle e is set equal to the value of θ′, and the method further computes a new value of the distance w by calculating the Equations (5) and (6) with the rotation angle θ, and then proceeds to step S312 again. If the ratio of the difference between θ and θ′ (i.e., Δθ) to the value θ is not smaller than the predetermined value, the method proceeds to step S316, and the value of the rotation angle θ is set equal to the value of θ′ considered as a convergent value. Afterward, the rotation angle of the vertebra is obtained.

According to an embodiment of the present invention, a method for measuring vertebral axial rotation comprises: obtaining an image, such as an anteroposterior view of an X-ray image, of a vertebra to be measured; determining centers of ellipses of pedicles of the vertebra projected on the image; measuring a distance between the centers of the ellipses; measuring a distance between a center of one of the pedicles and a medial axis of the vertebra; obtaining at least one shape parameter of the vertebra; and calculating an axial rotation angle of the vertebra according to the shape parameter and the measured distances.

According to an embodiment of the present invention, the method further comprises calibrating the axial rotation angle of the vertebra by calculating a trigonometric relationship of a shift distance between different vertebras projected on the image and an incident direction of an X-ray beam.

According to an embodiment of the present invention, the method further comprises displaying the image in the electronic format on a display or first transforming the image in the non-electronic format, such as a film or a picture, into the electronic format and displaying the same on a display.

According to an embodiment of the present invention, wherein the shape parameter of the vertebral is about half of a distance between the centers of the pedicles divided by the distance between a center of one of the pedicles and a medial axis of the vertebra or the shape parameter of the vertebra is a statistical mean value of the same vertebra of a plurality of bodies. According to an embodiment of the present invention, the shape parameter of the vertebra is determined according to an image generated by a computed tomography scanner or a nuclear magnetic resonance scanner.

According to an embodiment of the present invention, wherein the ellipses of pedicles of the vertebra projected on the image are identified by an image segmentation technique.

According to an embodiment of the present invention, wherein each center of the pedicles is obtained according to a midpoint of a major axis of the ellipse, an arithmetic mean value of coordinates of all pixels of each ellipse or an arithmetic mean value of coordinates of all boundary pixels of each ellipse after a boundary of each ellipse is thinned.

According to an embodiment of the present invention, the desired coordinates or distances on the image in the non-electronic format are calculated by an operator.

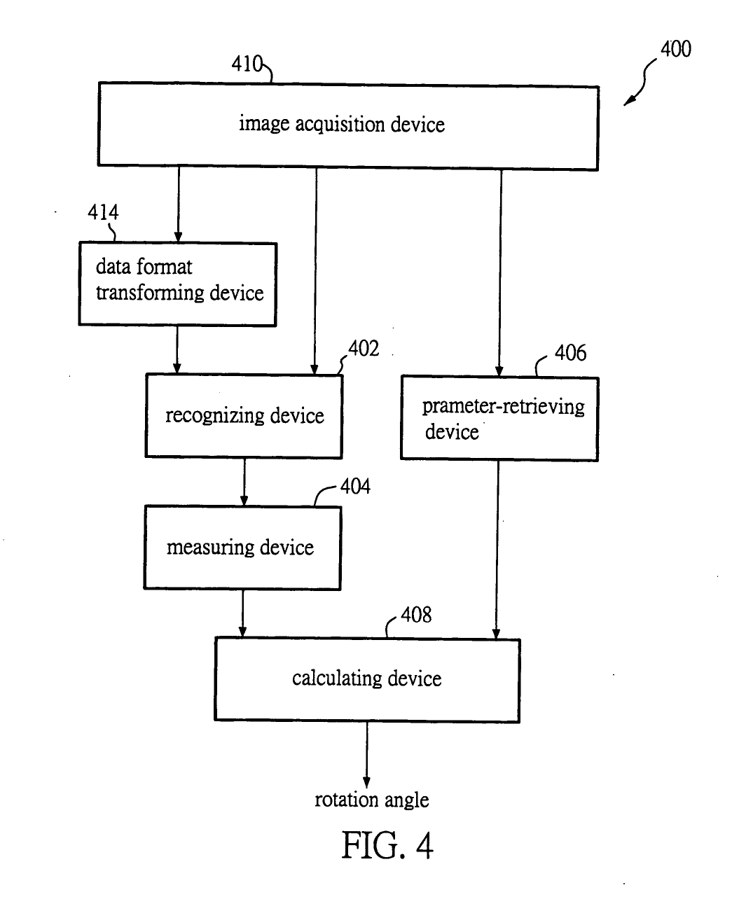

Please refer to FIG. 4. FIG. 4 is a functional block diagram of the apparatus 400 according to an embodiment of the present invention. The medical apparatus 400 comprises a recognizing device 402 for recognizing centers of ellipses of pedicles of the vertebra projected on the image; a measuring device 404 for measuring a distance between the centers of the ellipses and a distance between a center of one of the pedicles and a medial axis of the vertebra; a parameter-retrieving device 406 for retrieving at least one shape parameter of the vertebra; and a calculating device 408 for calculating an axial rotation angle of the vertebra according to the shape parameter and the measured distances.

According to an embodiment of the present invention, the apparatus 400 further comprises an image-acquisition devices 410. According to an embodiment of the present invention, the medical apparatus 400 further comprises data-format-transforming device 414. In the present embodiment, the image-acquisition device 410 may be a computed tomography scanner, a nuclear magnetic resonance scanner or an X-ray machine for generating a digital image or a non-digital image on a film or a picture. The generated digital image is directly transmitted to the recognizing device 402, and the non-digital image is transmitted to data-format-transforming device 414 for transforming into a digital image and then outputted to the recognizing device 402. The recognizing device 402 determines centers of ellipses of pedicles of the vertebra projected on the image as depicted in FIG. 1a and FIG. 1b according to an image slicing technique.

According to an embodiment of the present invention, the apparatus 400 further comprises a calibrating device for calibrating the axial rotation angle of the vertebra by calculating a trigonometric relationship of a shift distance between different vertebras projected on the image and an incident direction of an X-ray beam.

According to an embodiment of the present invention, the recognizing device 402 may thin the boundary of an ellipse, and calculate the arithmetic mean value of coordinates of all the pixels included in the boundary, and then the arithmetic mean value is utilized to be the location of the center of the ellipse. It should be noted that other methods, such as the method of utilizing the arithmetic mean value of coordinates of all the pixels of the ellipse to be the center of the ellipse and the method of utilizing the midpoint of the major axis of the ellipse to be the center of the ellipse, may be applied to the present invention.

According to an embodiment of the present invention, the measuring device 404 evaluates the distance between the centers and evaluates the distance between the center of the pedicle at the convex side and a medial axis of the vertebral body. The parameter-retrieving device 406 is utilized to output a shape parameter η of the vertebral body to the calculating device 408. Then, the calculating device 408 calculates the rotation angle θ of the vertebral axial according to the method recited in FIG. 3 with the shape parameter η and the measured distances. The parameter-retrieving device 406 is capable of utilizing the image from the image-acquisition device 410 to compute the shape parameter η according to the equation A̅D̅/ O̅D̅=η. According to an embodiment of the present invention, Table 1 mentioned above is stored in the parameter-retrieving device 406. Consequently, the parameter-retrieving device 406 is capable of determining the value of ∠AOD by using Table 1 and then computing the shape parameter η according to the equation ∠AOD=tan⁻¹ η. It should be noted that the recognizing device 402, the measuring device 404, the parameter obtaining device 406 and the calculating device 408 may be substantial circuits or program modules stored and executed by an operation terminal computer, a central processing host or a Personal Digital Assistant (PDA).

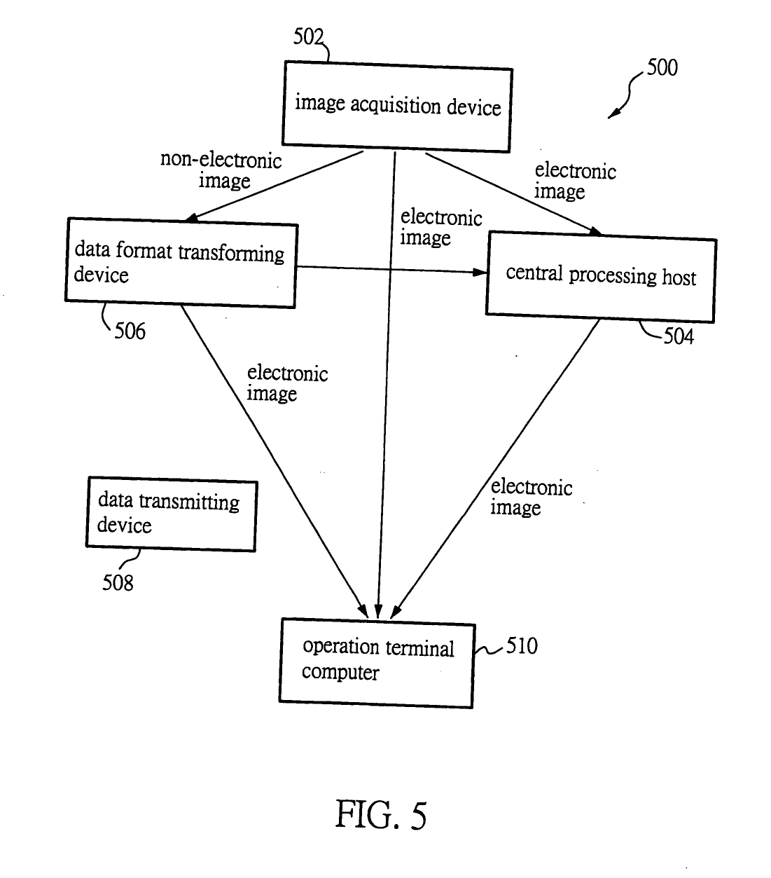

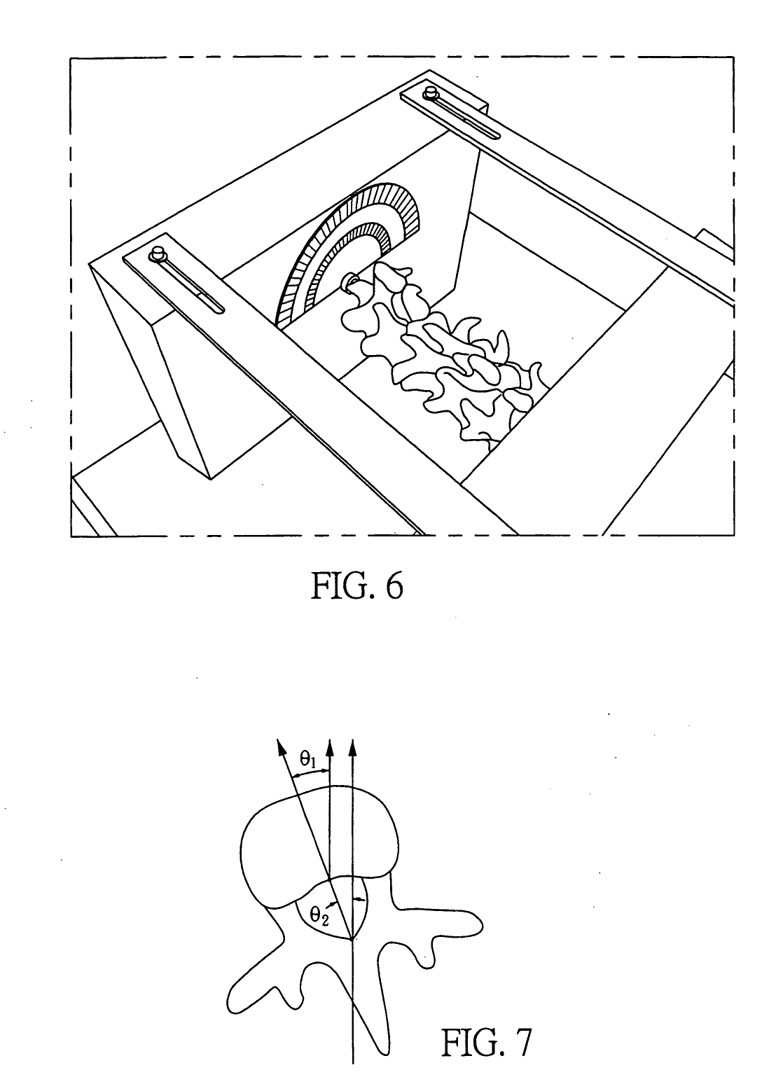

Please refer to FIG. 5. FIG. 5 is a schematic diagram illustrating the arrangement of an apparatus 500 according to an embodiment of the present invention. The apparatus 500 comprises an image-acquisition device 502, for example but not limited to an X-ray machine, a C-arm or a scanner, for obtaining an electronic-formatted (digital) or non-electronic-formatted (non-digital) X-ray image; and at least one operation terminal computer 510 storing a computer program. The computer program may be a single software package or a part of analyzing software for performing the above-mentioned methods of the present invention. The computer program can be stored on a machine-readable medium and executed by a computer, a PDA, or other machines. Examples of a machine-readable medium include recordable-type medium such as a floppy disc, a hard disc drive, a RAM and CD-ROMs and transmission-type medium such as digital and analog communication links.

According to an embodiment of the present invention, the apparatus 500 comprises a central processing host 504 for performing the above-mentioned methods of the present invention. It should be noted that the arrangement of these functions is various according to the present invention. Even all functions may be processed by one of the central processing host 504 and the operation terminal computer 510. In the present invention, the digital images outputted by the image-acquisition device 502 may be transmitted to the central processing host 504 and then transmitted to the operation terminal computer 510. However, the operation terminal computer 510 may directly access the digital images stored in the central processing host 504.

According to an embodiment of the present invention, the apparatus 500 further comprises a data-format-transforming device 506, such as a digitizer, backlight digitizer, or light box, for transforming non-digital images (e.g., X-ray films, pictures, and films) outputted by the acquisition device 502 into a digital image and then transmitting the digital images to the central processing host 504 or the operation terminal computer 510.

According to an embodiment of the present invention, the apparatus 500 further comprises a data-transmitting device 508, such as a wireless network, a wireless communication device, a physical network, a telephone line, a cable, a portable disk, a disk, an optical disk, a PDA, or a film folder, for transmitting the digital or non-digital images.

Please refer to FIG. 6. FIG. 6 is a schematic diagram of a cadaver spine rotation-fixation device, which has a rectangular polyethylene (PE) base of 28.5 cm×6 cm×20 cm on each side. The PE base has an open hole and a protractor attached to its center. A PE rod is inserted through the vertebral foramen, such that the lumbar spine is strung in series. Vertebrae are fixed to the rod with adhesive to permit coaxial rotation. A pointer is placed at the end of the rod. Therefore, when the lumbar segments rotate simultaneously, the pointer can indicate the protractor scale, and thus the lumbar segments can be rotated about a predetermined angle. However, the precise rotation angle of the lumbar segments shall be measured based on the CT image.

The upper left and right side of the PE base have two screw holes. Two acrylic rods having grooves at each end of the rods are fixed in the top of the base stage with screws. When the screws lock the grooves, the spinal rotation-fixation device is more stable. The spinal rotation-fixation device is placed on a wooden board, which supports the device and avoids any change in rotation state when transferring between X-rays and CT scans.

Before taking an image, the spinous process is set facing upward, and the pointer is aligned with 0 on the protractor. The lumbar spine is rotated gradually from 0 to 30 degrees at an increment of 5 degrees, to achieve a total of seven rotational states. At each state, one X-ray and CT image is taken. For X-rays, standard AP radiographs are taken. In the present embodiment, the distance between the X-ray tube and the film is set to 100 cm, as in actual clinical work. However, the distance between the X-ray tube and the film is not limited to 100 cm. In the present embodiment, the primary beam of the X-ray is aimed at the spinous process L3. The effect τ of the calculated rotation angle caused by the displacement of the spinal segment is represented as:

$$tan τ = \frac{\begin{matrix}the shift distance on horizontal \\ or vertical direction ( cm )\end{matrix}}{\begin{matrix}the distance between the \\ X \text{-} ray tube and the film ( cm )\end{matrix}} \qquad Equation ( 7 )$$

Some technical literature points out that the effect caused by the shift on the plane of the film could be neglected. People skilled in the art can easily calculate the rotation angle according to Equation (7). The increase or decrease of the distance between the X-ray tube and the film only changes the magnification and does not affect the resulting rotation measurement.

Please note that the protractor angle is only a reference for simulating the lumbar segments in various axial rotation states. Additionally, when segments are fixed on the PE axle, five spinous processes may not be completely aligned. Consequently, actual initial angles of the segments are only very close to 0 when the pointer is aligned with 90 degrees on the protractor. Thus, the actual segment rotation angle is confirmed on CT scans.

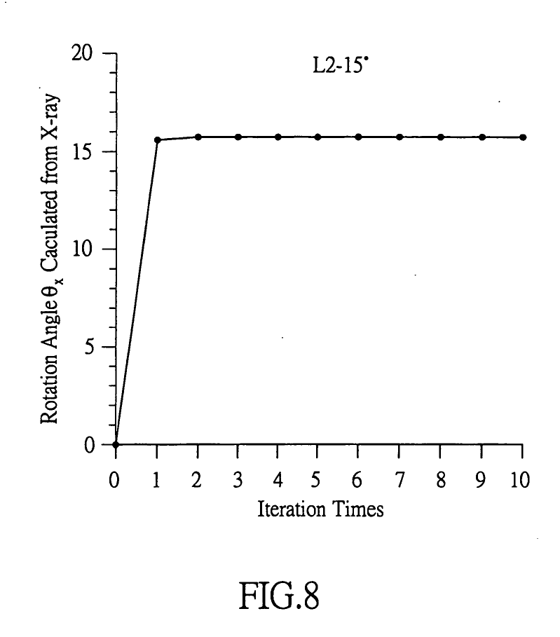

FIG. 7 illustrates how to measure actual rotation angles with CT images. Based on a CT image of the vertebra waist cutting through the pedicles, this work connects point H depicted in FIG. 1b and the vertebral body center, and the rotation angle θ₁ is identical to that used by Aaro et al., i.e., θ₂.

Based on partial damage of L5, the vertebral contour on the X-ray image is unidentifiable, and therefore, the rotation angle is not obtained. Consequently, only four lumbar segments (L1-L4) are assessed.

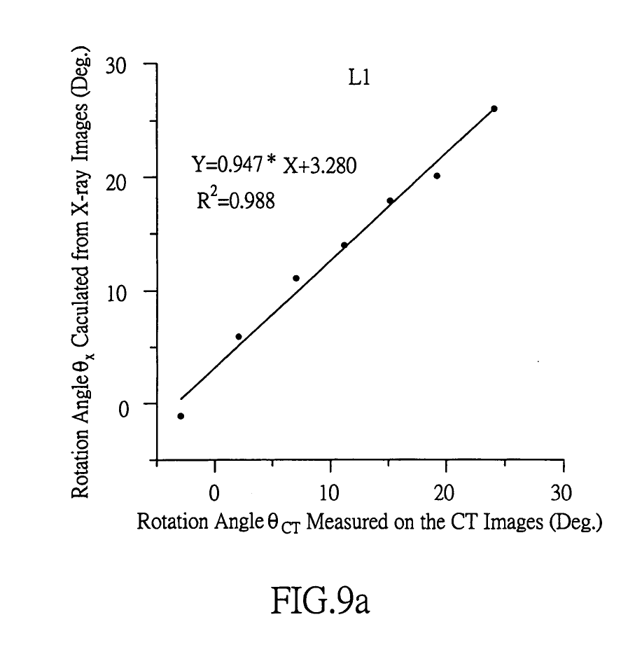

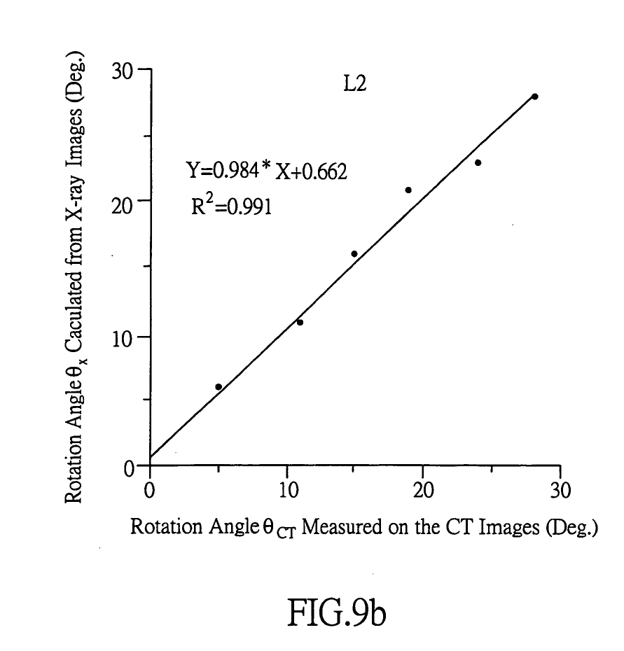

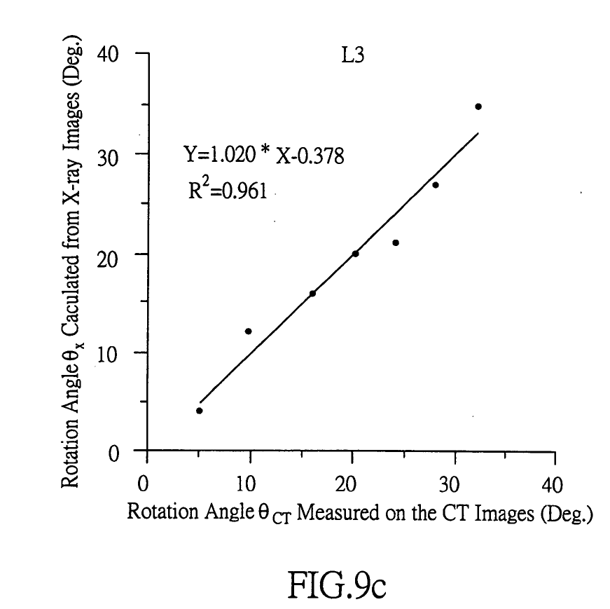

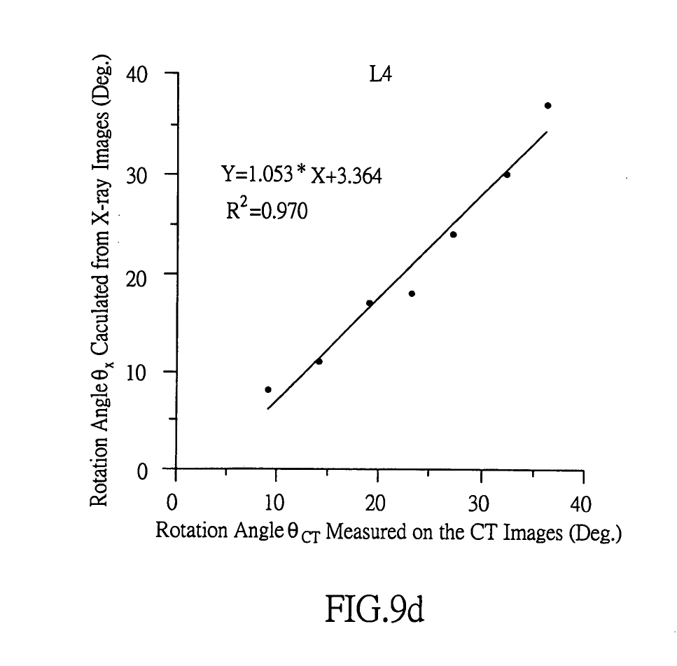

After marking the necessary anatomical landmarks on the X-ray image of four lumbar segments, a computer program based on the proposed equations is developed to determine the rotation angle. When the rotation angle of L2 depicted on FIG. 8 measured on a CT scan is 15 degrees, the angle, by the current method, rapidly converges to 15.7 degrees after 10 iterations. FIG. 9a to FIG. 9d are curves illustrating the relation between the actual rotation angle θ_{CT}, measured from CT images, and the rotation angle θ_{X}, estimated based on X-ray images of the four vertebrae L1-L4. For every vertebra, the calculated value θ_{X} and standard value θ_{CT} are strongly correlated, with R² of 0.988, 0.991, 0.961 and 0.970. FIG. 9 demonstrates the high correlation between the calculated value θ_{X} and standard value θ_{CT} during the rotation of each vertebra segment. In addition, the error of the calculation does not increase when the rotation angle increases from 0 degree to 30 degrees.

According to the method of the present invention, a rapid, easy and precise measurement of a rotation angle of a vertebral axial is obtained.

Although the technical contents and features of the present invention have been illustrated above, variations and modifications of the present invention without departing from the teachings and disclosure of the present invention can be made by those skilled in the art. Therefore, the protective scope of the present invention is not limited to the disclosure of the embodiments, but includes the variations and modifications without departing from the present invention, which is contemplated by the following claims.

What is claimed is:

1. A method for measuring vertebral axial rotation comprising:
obtaining an image of a vertebra to be measured;
determining centers of ellipses of pedicles of the vertebra projected on the image;
measuring a distance between the centers of the ellipses;
measuring a distance between a center of one of the pedicles and a medial axis of the vertebra;
obtaining at least one shape parameter of the vertebra; and
calculating an axial rotation angle of the vertebra according to the shape parameter and the measured distances.

2. The method of claim 1, wherein the shape parameter of the vertebral is about half of a distance between the centers of the pedicles divided by the distance between a center of one of the pedicles and a medial axis of the vertebra.

3. The method of claim 1, wherein the image is an anteroposterior view of an X-ray image.

4. The method of claim 3 further comprising:
calibrating the axial rotation angle of the vertebra by calculating a trigonometric relationship of a shift distance between different vertebras projected on the image and an incident direction of an X-ray beam.

5. The method of claim 1, wherein the image is in an electronic format or a non-electronic format comprising a film or a picture.

6. The method of claim 5, further comprising:
displaying the image in the electronic format on a display.

7. The method of claim 5 further comprising:
transforming the image in the non-electronic format into the electronic format and displaying the same on a display.

8. The method of claim 1, wherein the ellipses of pedicles of the vertebra projected on the image are identified by an image segmentation technique.

9. The method of claim 5, wherein desired coordinates or distances on the image in the non-electronic format are calculated by an operator.

10. The method of claim 8, wherein each center of the pedicles is obtained according to a midpoint of a major axis of the ellipse.

11. The method of claim 8, wherein each center of the pedicles is obtained according to an arithmetic mean value of coordinates of all pixels of each ellipse.

12. The method of claim 8, wherein each center of the pedicles is obtained according to an arithmetic mean value of coordinates of all boundary pixels of each ellipse after a boundary of each ellipse is thinned.

13. The method of claim 1, wherein the shape parameter of the vertebra is a statistical mean value of the same vertebra of a plurality of bodies.

14. The method of claim 1, wherein the shape parameter of the vertebra is determined according to an image generated by a computed tomography scanner or a nuclear magnetic resonance scanner.

15. The method of claim 1, wherein the axial rotation angle of the vertebra is calculated by an iteration process.

16. A machine-readable medium embodied thereon a program configured to measure vertebral axial rotation, the program comprising code segments for causing a machine to:
obtain an image of a vertebra to be measured;
determine centers of ellipses of pedicles of the vertebra projected on the image;
measure a distance between the centers of the ellipses;
measure a distance between a center of one of the pedicles and a medial axis of the vertebra;
obtain at least one shape parameter of the vertebra; and
calculate an axial rotation angle of the vertebra according to the shape parameter and the measured distances.

17. The machine readable medium of claim 16, wherein the program further comprises code segments for causing the machine to:
calibrate the axial rotation angle of the vertebra by calculating a trigonometric relationship of a shift distance between different vertebras projected on the image and an incident direction of an X-ray beam.

18. The machine readable medium of claim 16, wherein the program further comprises code segments for causing the machine to:
display the image in the electronic format on a display.

19. The machine readable medium of claim 16, wherein the program further comprises code segments for causing the machine to:
transform the image in the non-electronic format into the electronic format and displaying the same on a display.
